(19) United States
(12) Patent Application Publication
(10) Pub. No.: US 2015/0087856 A1
(43) Pub. Date: Mar. 26, 2015

(54) MELANIN PRODUCTION INHIBITOR

(71) Applicant: TAKASAGO INTERNATIONAL CORPORATION, Ota-ku, Tokyo (JP)

(72) Inventors: Toshiaki Narukami, Ota-ku (JP); Eiko Tamai, Yokohama-shi (JP)

(73) Assignee: TAKASAGO INTERNATIONAL CORPORATION, Ota-ku, Tokyo (JP)

(21) Appl. No.: 14/386,553

(22) PCT Filed: Apr. 2, 2013
(86) PCT No.: PCT/JP2013/060073

§ 371 Date: Sep. 19, 2014

(30) Foreign Application Priority Data
Apr. 2, 2012 (JP) 2012-083764

(51) Int. Cl. A61K 8/34; C07C 69/675; C11B 9/00; C07C 69/013; A61K 8/37; A61Q 19/02; C07C 35/12; C07C 43/196

(52) U.S. Cl. CPC A61K 8/34; C07C 35/12; C07C 69/675; C07C 43/196; C07C 69/013; A61K 8/37; A61K 8/345; A61Q 19/02; C11B 9/0034; C07C 2101/14; A61K 2800/782
USPC 560/188; 568/829; 568/670; 560/174

(57) ABSTRACT

The present invention provides a melanin production inhibitor containing a compound having a (2-isopropyl-5-methyl)cyclohexyl group or a (2-isopropenyl-5-methyl)cyclohexyl group.

### TECHNICAL FIELD

The present invention relates to a melanin production inhibitor for inhibiting darkening of the skin due to ultraviolet rays or the like, and a skin-whitening cosmetic which is excellent in skin-whitening effect and is highly safe.

### BACKGROUND ART

When exposed to ultraviolet rays contained in sunlight, ultraviolet lamp, or the like, the skin loses the glow, fine texture, moisture, and the like. Especially when the dermis is damaged by ultraviolet rays, wrinkles and sagging are caused, which cause the so-called photoaging.

Active oxygen generated by exposure to ultraviolet rays and various factors released from cells of the skin because of the influence of the active oxygen enhance tyrosinase activity in melanocytes. Melanin, which has a relation to the color tone of the skin, is produced by oxidation of tyrosine with tyrosinase in melanocytes. It is thought that activation of tyrosinase by ultraviolet rays results in excessive production of melanin, and the melanin is transferred to epidermal cells, so that the color tone of the skin changes and darkens.

Hence, it is known that melanin production inhibition is effective in obtaining skin-whitening effect.

Known active ingredients include ascorbic acid, Kojic acid, arbutin, ellagic acid, 4-alkylresorcinols, and derivatives thereof, as well as various plant extracts. Besides these active ingredients, the following and the like are known. Specifically, a high melanin production-inhibitory effect is exhibited when a compound which is an ester of menthol with a long-chain unsaturated fatty acid having 18 or more carbon atoms is blended with an active ingredient such as an agent for inhibiting a transmitter produced by keratinocytes, an antioxidant, an antiinflammatory, a polymer compound, or a polyvalent alcohol, because of a synergistic effect of each combination (Japanese Patent Application Publication No. 2007-161591). Meanwhile, menthol inhibits the activity of tyrosinase, which is an enzyme involved in biosynthesis of melanin (Japanese Patent Application Publication No. 2006-16343).

### SUMMARY OF INVENTION

An object of the present invention is to provide a useful melanin production inhibitor.

In view of such circumstances, the present inventors have conducted intensive study. Consequently, the present inventors have found that a certain compound group has excellent melanin production-inhibitory effect.

Specifically, the present invention includes the following contents [1] to [7].

[1] A melanin production inhibitor comprising a compound having a (2-isopropyl-5-methyl)cyclohexyl group or a (2-isopropenyl-5-methyl)cyclohexyl group.
[2] The melanin production inhibitor, wherein the compound having a (2-isopropyl-5-methyl)cyclohexyl group or a (2-isopropenyl-5-methyl)cyclohexyl group is a compound of the following formula (I):

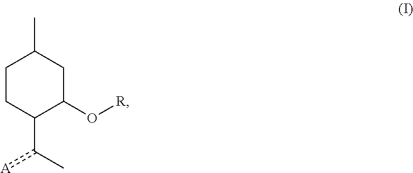

wherein R represents a hydrogen atom, an optionally substituted linear or branched alkyl group having 1 to 6 carbon atoms, an optionally substituted linear or branched alkenyl group having 2 to 6 carbon atoms, or an optionally substituted linear or branched acyl group having 1 to 6 carbon atoms, the dashed lines represent a single bond or a double bond, and when the dashed lines are a single bond, A represents CH₃, while when the dashed lines are a double bond, A represents CH₂.
[3] The melanin production inhibitor according to the above-described [1] or [2], wherein

the compound having a (2-isopropyl-5-methyl)cyclohexyl group or a (2-isopropenyl-5-methyl)cyclohexyl group is at least one compound selected from
5-methyl-(2-isopropenyl)cyclohexanol,
3-(2-isopropyl-5-methylcyclohexyloxy) propane-1,2-diol,
3-(2-isopropyl-5-methylcyclohexyloxy) propan-1-ol,
2-isopropyl-5-methylcyclohexyl 3-hydroxybutyrate,
2-(2-(2-isopropyl-5-methylcyclohexyloxy) ethoxy) ethanol,
1-(2-isopropyl-5-methylcyclohexyloxy)propan-2-ol, and
2-isopropyl-5-methylcyclohexyl 3-oxobutyrate.
   [4] A fragrance composition comprising the melanin production inhibitor according to the above-described [1] to [3].
   [5] A skin-whitening agent comprising the melanin production inhibitor according to the above-described [1] to [3].
   [6] A cosmetic comprising the melanin production inhibitor according to the above-described [1] to [3].
   [7] A skin external agent comprising the melanin production inhibitor according to the above-described [1] to [3].

The present invention provides fragrance compositions, skin-whitening cosmetics, and the like using the melanin production inhibitor having high inhibitory effect.

### DESCRIPTION OF EMBODIMENTS

Hereinafter, the present invention will be described in detail.

The melanin production inhibitor of the present invention comprises a compound having a (2-isopropyl-5-methyl)cyclohexyl group or a (2-isopropenyl-5-methyl)cyclohexyl group.

The compound preferably has a structure represented by the following formula (I):

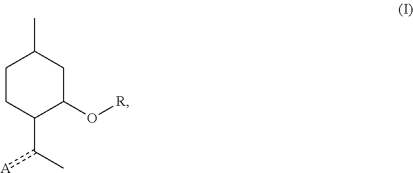

wherein R represents a hydrogen atom, an optionally substituted linear or branched alkyl group having 1 to 6 carbon atoms, an optionally substituted linear or branched alkenyl group having 2 to 6 carbon atoms, or an optionally substituted linear or branched acyl group having 1 to 6 carbon atoms. In addition, the dashed lines represent a single bond or a double bond, and when the dashed lines are a single bond, A represents CH₃, while when the dashed lines are a double bond, A represents CH₂.

The alkyl group having 1 to 6 carbon atoms may be a methyl group, an ethyl group, a propyl group, a butyl group, a pentyl group, a hexyl group, or the like, and is preferably an alkyl group having 3 or 4 carbon atoms. These groups may be linear or branched.

Meanwhile, the alkenyl group having 2 to 6 carbon atoms may be an ethenyl group, a propenyl group, a butenyl group, a pentenyl group, a hexenyl group, or the like, and is preferably an alkenyl group having 3 or 4 carbon atoms. These groups may be linear or branched.

Moreover, the acyl group having 1 to 6 carbon atoms may be a formyl group, an acetyl group, a propionyl group, a butanoyl group, a pentanoyl group, a heptanoyl group, or the like, and is preferably an acyl group having 3 or 4 carbon atoms. These groups may be linear or branched.

The above-described alkyl groups, alkenyl groups, and acyl groups may be substituted, and the substituents may be hydroxyl groups, lower alkoxy groups, oxo groups, and the like. Here, the lower alkoxy groups have 1 to 8 carbon atoms, preferably 1 to 6 carbon atoms, and more preferably 1 to 4 carbon atoms. Examples of the lower alkoxy groups include methoxy groups, ethoxy groups, propoxy groups, butoxy groups, pentoxy groups, hexyloxy groups, heptyloxy groups, octyloxy groups, cyclobutoxy groups, cyclopentoxy groups, and cyclohexyloxy groups.

Specific compounds of formula (I) include
5-methyl-(2-isopropyl)cyclohexanol,
5-methyl-(2-isopropenyl)cyclohexanol,
3-(2-isopropyl-5-methylcyclohexyloxy)propane-1,2-diol,
3-(2-isopropyl-5-methylcyclohexyloxy)propan-1-ol,
2-isopropyl-5-methylcyclohexyl 3-hydroxybutyrate,
2-(2-(2-isopropyl-5-methylcyclohexyloxy)ethoxy)ethanol,
1-(2-isopropyl-5-methylcyclohexyloxy)propan-2-ol,
2-isopropyl-5-methylcyclohexyl 3-oxobutyrate, 2-(2-hydroxypropane-2-yl)-5-methylcyclohexanol,
2-(2-isopropyl-5-methylcyclohexyloxy)ethanol,
3-(2-isopropyl-5-methylcyclohexyloxy)-2-methylpropane-1,2-diol,
1-(2-hydroxy-4-methylcyclohexyl)ethanone,
2-isopropyl-5-methylcyclohexyl 2,2-dihydroxyacetate,
2-isopropyl-5-methylcyclohexyl acetate,
4-(2-isopropyl-5-methylcyclohexyloxy)-4-oxobutanoic acid,
5-(2-isopropyl-5-methylcyclohexyloxy)-5-oxopentanoic acid, and the like.

Of these compounds, preferred are
5-methyl-(2-isopropenyl)cyclohexanol,
3-(2-isopropyl-5-methylcyclohexyloxy)propane-1,2-diol,
3-(2-isopropyl-5-methylcyclohexyloxy)propan-1-ol,
2-isopropyl-5-methylcyclohexyl 3-hydroxybutyrate,
2-(2-(2-isopropyl-5-methylcyclohexyloxy)ethoxy)ethanol,
1-(2-isopropyl-5-methylcyclohexyloxy)propan-2-ol,
2-isopropyl-5-methylcyclohexyl 3-oxobutyrate, and the like.

As the compound having a (2-isopropyl-5-methyl)cyclohexyl group or a (2-isopropenyl-5-methyl)cyclohexyl group, a compound produced by a known method or a commercially available compound can be used. Moreover, each compound may be used in an optically active form or as a racemic or diastereomeric mixture.

The compound having a (2-isopropyl-5-methyl)cyclohexyl group or a (2-isopropenyl-5-methyl)cyclohexyl group has melanin production-inhibitory action. A melanin production inhibitor can be obtained by preparing a composition in which the compound is blended. The melanin production inhibitor of the present invention can be used as a mixture with a known melanin production inhibitor (for example, pantetheine-s-sulfonic acid, isoferulic acid, ascorbic acid, derivatives thereof, arbutin, Kojic acid, linoleic acid, ellagic acid, glycyrrhizic acid, licorice extract, or the like).

Moreover, by being mixed with various bases, additives, and the like, the melanin production inhibitor of the present invention can be used as, for example, fragrance compositions, skin-whitening agents, skin-whitening cosmetics, cosmetics, skin external agents, skin-whitening emulsion cosmetics, skin-whitening creams, skin-whitening lotions, skin-whitening oil-based cosmetics, skin-whitening packs, skin-whitening foundations, and the like. Further, it is possible to provide a synergistic effect by mixing with another skin-whitening agent or the like. In the above-described fragrance composition or the like, the blended amount of the compound having a (2-isopropyl-5-methyl)cyclohexyl group or a (2-isopropenyl-5-methyl)cyclohexyl group can be set, as appropriate, by those skilled in the art according to a desired melanin production-inhibitory action or the like, and is preferably 0.00001 to 10% by mass and further preferably 0.0001 to 1% by mass, based on the total amount of a finished product.

The melanin production inhibitor, the cosmetic, and the like of the present invention can be produced according to commonly employed pharmaceutical production methods.

The melanin production inhibitor and the cosmetic of the present invention can contain oils, fats, and the like such as vegetable oils, higher fatty acids, higher alcohols, silicones, anionic surfactants, cationic surfactants, amphoteric surfactants, nonionic surfactants, preservatives, saccharides, metal ion-sequestering agents, polymers such as water-soluble polymers, thickeners, powder components, ultraviolet absorbers, ultraviolet screening agents, humectants such as hyaluronic acid, fragrances, pH adjusters, and the like. The melanin production inhibitor and the cosmetic of the present invention can also contain other active medical ingredients and bioactive components such as vitamins, skin activators, blood circulation promoters, indigenous bacteria-controlling agents, active oxygen scavengers, antiinflammatories, other skin-whitening agents, and microbicides.

Examples of the oils, fats, and the like include liquid fatty oils such as camellia oil, evening primrose oil, macadamia nut oil, olive oil, rapeseed oil, corn oil, sesame oil, jojoba oil, germ oil, wheat germ oil, triglycerides, and glyceryl trioctanoate, solid fats such as cacao butter, coconut oil, hydrogenated coconut oil, palm oil, palm kernel oil, Japan wax, Japan wax kernel oil, hydrogenated oils, and hydrogenated castor oil, waxes such as beeswax, candelilla wax, cotton wax, rice bran wax, lanolin, acetylated lanolin, liquid lanolin, and sugarcane wax, liquid paraffin, squalene, squalane, microcrystalline wax, and the like.

Examples of the higher fatty acids include lauric acid, myristic acid, palmitic acid, stearic acid, oleic acid, linoleic acid, linolenic acid, docosahexaenoic acid (DHA), eicosapentaenoic acid (EPA), and the like.

Examples of the higher alcohols include linear alcohols such as lauryl alcohol, stearyl alcohol, cetyl alcohol, and cetostearyl alcohol; branched alcohols such as monostearyl glycerin ether, lanolin alcohol, cholesterol, phytosterols, and octyldodecanol; and the like.

Examples of the silicones include linear polysiloxanes such as dimethyl polysiloxane and methyl phenyl polysiloxane; and cyclic polysiloxanes such as decamethyl polysiloxane. Examples of the anionic surfactants include fatty acid salts such as sodium laurate; higher-alkyl sulfate salts such as sodium lauryl sulfate; alkyl ether sulfate salts such as POE-lauryl sulfate triethanolamine salt; N-acylsarcosine acid; sulfosuccinic acid salts; N-acylamino acid salts; and the like. Examples of the cationic surfactants include alkyltrimethylammonium salts such as stearyltrimethylammonium chloride; benzalkonium chloride; benzethonium chloride; and the like. Examples of the amphoteric surfactants include betaine-based surfactants such as alkyl betaines and amide betaines; and the like. Examples of the nonionic surfactants include sorbitan fatty acid esters such as sorbitan monooleate; and hydrogenated castor oil derivatives.

Examples of the preservatives include methyl paraben, ethyl paraben, and the like.

Examples of the metal ion-sequestering agents include disodium ethylenediaminetetraacetate, edetic acid, and edetic acid salts such as edetic acid sodium salts.

Examples of the polymers include gum arabic, gum tragacanth, galactan, guar gum, carrageenan, pectin, agar, quince seeds, dextran, pullulan, carboxymethyl starch, collagen, casein, gelatin, methyl cellulose, methyl hydroxypropyl cellulose, hydroxyethyl cellulose, carboxymethyl cellulose sodium (CMC), sodium alginate, vinyl polymers such as carboxyvinyl polymers (CARBOPOL and the like), bentonite, and the like.

Examples of the thickeners include carrageenan, gum tragacanth, quince seeds, casein, dextrin, gelatin, CMC, hydroxyethyl cellulose, hydroxypropyl cellulose, carboxyvinyl polymers, guar gum, xanthan gum, and the like.

Examples of the powder components include talc, kaolin, mica, silica, zeolite, polyethylene powder, polystyrene powder, cellulose powder, inorganic white pigments, inorganic reddish pigments, pearl pigments such as titanium oxide-coated mica, titanium oxide-coated talc, and colored titanium oxide-coated mica, and organic pigments such as Red No. 201 and Red No. 202.

Examples of the ultraviolet absorbers include p-aminobenzoic acid, phenyl salicylate, isopropyl p-methoxycinnamate, octyl p-methoxycinnamate, 2,4-dihydroxybenzophenone, and the like.

Examples of the ultraviolet screening agents include titanium oxide, talc, carmine, bentonite, kaolin, zinc oxide, and the like.

Examples of the humectants include polyethylene glycol, propylene glycol, dipropylene glycol, 1,3-butylene glycol, glycerin, diglycerin, polyglycerin, xylitol, maltitol, maltose, sorbitol, glucose, fructose, sucrose, lactose, chondroitin sulfate sodium salt, sodium hyaluronate, sodium lactate, pyrrolidone carboxylic acid, cyclodextrin, ceramide, derivatives thereof, and the like.

Examples of the active medical ingredients include vitamins including vitamin A such as vitamin A oil and retinol, vitamin B2 such as riboflavin, vitamin B6 such as pyridoxine hydrochloride, vitamin C such as L-ascorbic acid, L-ascorbic acid phosphate ester, L-ascorbic acid monopalmitate, L-ascorbic acid dipalmitate, and L-ascorbic acid-2-glucoside, pantothenic acids such as calcium pantothenate, vitamin D such as vitamin D2 and cholecalciferol; vitamin E such as α-tocopherols, tocopheryl acetate, and DL-α-tocopheryl nicotinate, and the like.

A fragrance composition can be prepared from the melanin production inhibitor of the present invention by mixing with one or two or more of commonly used fragrance components.

The “commonly used fragrance components” herein include various synthetic fragrances, naturally occurring essential oils, synthetic essential oils, citrus oils, animal fragrances, and the like. For example, it is possible to use a wide variety of fragrance components such as those described in “Perfume and Flavor Chemicals (Aroma Chemicals) 1, 2” (Steffen Arctender (1969)), “GOUSEI KOURYOU, KAGAKU TO SHOUHIN CHISHIKI (Synthetic Flavors and Fragrances, Chemistry and Product Knowledge)” (authored by Indo Motoichi, published on Mar. 6, 1996 by The Chemical Daily Co., Ltd.), or “Collection of Well-known Prior Arts (Flavors and Fragrances) Part I” (published on Jan. 29, 1999, by Japan Patent Office). Of these fragrance components, typical examples include a-pinene, limonene, cis-3-hexenol, phenylethyl alcohol, styralyl acetate, eugenol, rose oxide, linalool, benzaldehyde, muscone, Musk T (registered trademark of Takasago International Corporation), and Thesaron (registered trademark of Takasago International Corporation), as well as fragrances having cooling sensation effect such as menthol and derivatives having a menthane skeleton, and the like. The use of such a fragrance component in combination with the compound of the present invention makes it possible to improve the odor quality and note of the prepared fragrance, or provide a cooling sensation effect to the prepared fragrance.

In addition, multiple steps including tyrosinase-related steps, transfer to the epidermis, and the like are involved in the skin-whitening effect. Accordingly, it is possible to enhance the skin-whitening activity by using a mixture of multiple skin-whitening agents selected from various skin-whitening agents including the representative compounds having skin-whitening activity described in BACKGROUND ART.

Hereinafter, the present invention will be described more specifically by way of Examples. However, the present invention is not limited to these Examples.

### EXAMPLES

### Example 1

### Visual Evaluation Test for Melanin Production Inhibition in Pigment Cells

In plastic culture flasks (25 cm²), B-16 melanoma cells were seeded at 5×10⁴ per flask, and cultured in DMEM (Dulbecco's Modified Eagle's Medium) containing 10% serum [Nippon Suisan Kaisha, Ltd, trade name] in the presence of 5% carbon dioxide at a temperature of 37° C. On the next day, each test sample diluted with ethanol was added at concentrations in the medium of 3.13, 6.25, 12.5, 25, and 50 ppm, and the cells were cultured for further four days. After the culturing was finished, the medium was removed, and the cells were rinsed with phosphate buffered solution (hereinafter, referred to as PBS). Then, the cells were detached from each flask by using trypsin-containing PBS, and were harvested from the cell suspension by centrifugation. After the obtained cells were rinsed once with PBS, the whiteness of the sediment was visually observed. Compounds 2A and 2B were used as comparative materials. Table 1 shows the results.

TABLE 1
Visual Evaluation Test for Melanin Production
Inhibition

| | Concentration (ppm) | | | | |
|---|---|---|---|---|---|
| Compound | 50 | 25 | 12.5 | 6.25 | 3.13 |
| 1A | | +++ | ++ | − | |
| 1B | | | | ++ | + |
| 1C | | | +++ | ++ | − |
| 1D | | | +++ | ++ | − |
| 1E | | | ++ | + | − |
| 1F | | | +++ | ++ | |
| 1G | | | +++ | +++ | + |
| 1H | | | +++ | ++ | + |
| 1I | | | +++ | + | |
| 1J | | | +++ | + | |
| 1K | | | +++ | + | |
| 2A | +++ | ++ | + | − | |
| 2B | + | − | | | |
| Arbutin | | | +++ | ++ | + |

Activity: The activity was evaluated by using the following four grades.
−: The same as the solvent control (black)
+: Slightly different from the solvent control (dark gray)
++: Apparently different from the solvent control (light gray)
+++: Cells were not colored (white)

### Compounds:

1A: (1R,2S,5R)-5-methyl-2-isopropenylcyclohexanol
1B: 3-(2-isopropyl-5-methylcyclohexyloxy)propane-1,2-diol
1C: 3-(2-isopropyl-5-methylcyclohexyloxy)propan-1-ol
1D: 2-isopropyl-5-methylcyclohexyl 3-hydroxybutyrate
1E: 2-(2-(2-isopropyl-5-methylcyclohexyloxy)ethoxy)ethanol
1F: 1-(2-isopropyl-5-methylcyclohexyloxy)propan-2-ol
1G: 2-isopropyl-5-methylcyclohexyl 3-oxobutyrate
1H: 2-isopropyl-5-methylcyclohexanols
1I: (1S*,2S*,5R*)-2-isopropyl-5-methylcyclohexanol
1J: (1S*,2R*,5R*)-2-isopropyl-5-methylcyclohexanol
1K: (1R*,2R*,5R*)-2-isopropyl-5-methylcyclohexanol
2A: N-ethyl-5-methyl-2-isopropylcyclohexane carboxamide
2B: N,2,3-trimethyl-2-isopropylbutanamide

The results shown in Table 1 indicate that the compounds of the present invention were apparently excellent in melanin production-inhibitory activity in the visual evaluation test.

In addition, in the visual evaluation test, compounds 1C, 1D, 1F, and 1H were found to have almost the same activity as that of arbutin, which has been widely used as a substance having melanin-inhibitory activity, and further compound 1G was found to have higher activity than arbutin at 6.25 ppm.

### Example 2

### Quantification Test for Melanin Production Inhibition in Pigment Cells

In plastic culture flasks (25 cm²), B-16 melanoma cells were seeded at 5×10⁴ per flask, and cultured in DMEM containing 10% serum in the presence of 5% carbon dioxide at a temperature of 37° C. After culturing for three days, each test sample diluted with ethanol was added at a concentration in the medium of 6.25 ppm, and the cells were culture for further two days. After the culturing was finished, the medium was removed, and the cells were rinsed with phosphate buffered solution (hereinafter referred to as PBS). Then, the cells were detached from each flask by using trypsin-containing PBS, and harvested from the cell suspension by centrifugation. To the obtained cells, 2 ml of DMEM was added, and the cells were dispersed by gentle pipetting. Of the DMEM, 100 μl was diluted with DMEM to achieve 1×10⁴ to 5×10⁵ cells/ml. Then, the number of the cells was counted with Scepter™ 2.0 automated cell counter (Merck Millipore), and the cell growth ratio was determined. The cell growth ratio was determined by the following formula.

Cell growth ratio (%)=(number of cells of sample/number of cells of control)×100

The rest of the cell suspension was again centrifuged, and the medium was removed. After that, an operation in which the cells were rinsed with 1 ml of very cool 5% trichloroacetic acid and centrifuged was repeated three times, and further the cells were rinsed twice by adding 1 ml of ethanol/ethyl ether (3:1 volume ratio) and centrifuged. Finally, the cells were rinsed once with 1 ml of ethyl ether, centrifuged, and air-dried over night. To the dried cells, 2 N NaOH was added, and melanin in the cells was dissolved in a water bath of 60° C. After cooling, the absorbance at 415 nm was measured by using a microplate reader (BIO-RAD MICROPLATE READER Benchmark-). From the measured value, the amount of melanin per 10⁶ cells was calculated by conversion using a standard curve obtained by using synthesized melanin (NACALAI TESQUE, INC.), and the melanin inhibition ratio was determined. The melanin inhibition ratio was determined by the following formula:

Melanin inhibition ratio (%)=[(amount of melanin of control−amount of melanin of sample)/(amount of melanin of control)]×100

Table 2 shows the results of the cell growth ratio and the melanin inhibition ratio.

TABLE 2
Quantification Test for Melanin Production Inhibition
(concentrations were all 6.25 ppm)

| | Melanin production | |
|---|---|---|
| Compound | inhibition ratio | Cell growth ratio |
| 1B | 37.0% | 93.2% |
| 1C | 52.0% | 103.1% |
| 1D | 52.8% | 104.9% |
| 1E | 23.3% | 96.4% |
| 1G | 41.5% | 87.8% |
| 1H | 49.0% | 102.1% |
| Arbutin | 35.5% | 94.1% |

The results shown in Table 2 indicate that compounds 1B, 1C, 1D, 1G, and 1H of the present invention had higher melanin production-inhibitory activity than arbutin at 6.25 ppm in the quantification test for melanin production inhibition.

### Example 3

### Skin-Whitening Lotion

A skin-whitening lotion was prepared by dissolving the components shown in the following table at room temperature with stirring.

| | | Blended amount |
|---|---|---|
| Component | | (% by mass) |
| 1. | Glycerin | 10 |
| 2. | 1,3-Butylene glycol | 5 |
| 3. | Glucose | 2 |
| 4. | Ethanol | 5 |
| 5. | Carboxyvinyl polymer | 0.02 |
| 6. | Dipotassium glycyrrhizate | 0.1 |
| 7. | Sodium hyaluronate | 0.001 |
| 8. | 3-(2-isopropyl-5-methylcyclohexyloxy)propan- | 0.1 |
| | 1-ol | |
| 9. | Citric acid | 0.05 |
| 10. | Sodium citrate | 0.1 |
| 11. | Potassium hydroxide | 0.01 |
| 12. | Purified water | the balance |

The obtained skin-whitening lotion was better in skin-whitening effect than a lotion of a formulation in which the compound of the present invention was not blended, and was also good in storage stability.

### Example 4

### Skin-Whitening Cream

A skin-whitening cream was prepared by dissolving the components shown in the following table at room temperature with stirring.

| | | Blended amount |
|---|---|---|
| Component | | (% by mass) |
| 1. | Stearyl alcohol | 6 |
| 2. | Stearic acid | 2 |
| 3. | Squalene | 10 |
| 4. | Octyldodecanol | 5 |
| 5. | Olive oil | 5 |
| 6. | 1,3-Butylene glycol | 8 |
| 7. | Polyethylene glycol 1500 | 4 |
| 8. | POE(25) cetyl alcohol ether | 3 |
| 9. | Glyceryl monostearate | 2 |
| 10. | 2-Isopropyl-5-methylcyclohexyl | 0.1 |
| | 3-hydroxybutyrate | |
| 11. | Purified water | the balance |

The obtained skin-whitening cream was better in skin-whitening effect than a cream of a formulation in which the compound of the present invention was not blended, and was also good in storage stability.

### Example 5

### Skin-Whitening Pack

A skin-whitening pack was prepared by dissolving the components shown in the following table at room temperature with stirring.

| | | Blended amount |
|---|---|---|
| Component | | (% by mass) |
| 1. | Polyvinyl alcohol | 15 |
| 2. | Carboxymethyl cellulose | 5 |
| 3. | 1,3-Butylene glycol | 5 |
| 4. | Ethanol | 12 |
| 5. | 2-Isopropyl-5-methylcyclohexyl | 0.05 |
| | 3-oxobutyrate | |
| 6. | POE oleyl alcohol ether | 0.5 |
| 7. | Citric acid | 0.02 |
| 8. | Sodium citrate | 0.04 |
| 9. | Purified water | the balance |

The obtained skin-whitening pack was better in skin-whitening effect than a pack of a formulation in which the compound of the present invention was not blended, and was also good in storage stability.

1. A melanin production inhibitor comprising a compound having a (2-isopropyl-5-methyl)cyclohexyl group or a (2-isopropenyl-5-methyl)cyclohexyl group.

2. The melanin production inhibitor according to claim 1, wherein
the compound having a (2-isopropyl-5-methyl)cyclohexyl group or a (2-isopropenyl-5-methyl)cyclohexyl group has a structure represented by the following formula (I): 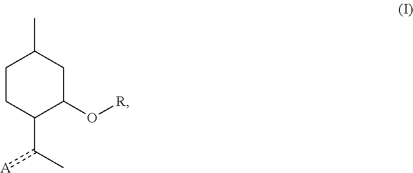
wherein R represents a hydrogen atom, an optionally substituted linear or branched alkyl group having 1 to 6 carbon atoms, an optionally substituted linear or branched alkenyl group having 2 to 6 carbon atoms, or an optionally substituted linear or branched acyl group having 1 to 6 carbon atoms, the dashed lines represent a single bond or a double bond, and when the dashed lines are a single bond, A represents CH₃, while when the dashed lines are a double bond, A represents CH₂.

3. The melanin production inhibitor according to claim 1, wherein
the compound having a (2-isopropyl-5-methyl)cyclohexyl group or a (2-isopropenyl-5-methyl)cyclohexyl group is at least one compound selected from
5-methyl-(2-isopropenyl)cyclohexanol,
3-(2-isopropyl-5-methylcyclohexyloxy)propane-1,2-diol,
3-(2-isopropyl-5-methylcyclohexyloxy)propan-1-ol,
2-isopropyl-5-methylcyclohexyl 3-hydroxybutyrate,
2-(2-(2-isopropyl-5-methylcyclohexyloxy)ethoxy)ethanol,
1-(2-isopropyl-5-methylcyclohexyloxy)propan-2-ol, and
2-isopropyl-5-methylcyclohexyl 3-oxobutyrate.

4. A fragrance composition comprising the melanin production inhibitor according to claim 1.

5. A skin-whitening agent comprising the melanin production inhibitor according to claim 1.

6. A cosmetic comprising the melanin production inhibitor according to claim 1.

7. A skin external agent comprising the melanin production inhibitor according to claim 1.
